(19) United States
(12) Patent Application Publication
(10) Pub. No.: US 2022/0031669 A1
(43) Pub. Date: Feb. 3, 2022

(54) Compositions for Broad Spectrum Topical Antimicrobials

(71) Applicants: Allen Gene HIRSH, Chevy Chase, MD (US); Cynthia S. Ricard, Gaithersburg, MD (US)

(72) Inventors: Allen Gene HIRSH, Chevy Chase, MD (US); Cynthia S. Ricard, Gaithersburg, MD (US)

(21) Appl. No.: 17/389,259

(22) Filed: Jul. 29, 2021

Related U.S. Application Data
(60) Provisional application No. 63/058,580, filed on Jul. 30, 2020.

(51) Int. Cl. A61K 31/427; A61K 47/20; A61K 47/22; A61K 47/18; A61K 31/166; A61K 31/7048; A61K 31/7052; A61K 31/727; A61K 38/16; A61K 31/4174; A61K 31/396; A61K 31/16; A61K 31/122; A61K 31/05; A61K 31/404

(52) U.S. Cl. CPC A61K 31/427; A61K 31/404; A61K 47/22; A61K 47/186; A61K 31/166; A61K 31/7048; A61K 31/7052; A61K 31/727; A61K 38/16; A61K 31/4174; A61K 31/396; A61K 31/16; A61K 31/122; A61K 31/05; A61K 47/20

(57) ABSTRACT

In response to the threat of global pandemics and bacterial species becoming increasingly resistant to antibiotic attack, this invention provides systems and methods for reducing the infectivity of bacteria and viruses in the human body by introducing onto the human body or into the human body or onto personal protective equipment or any combination thereof aqueous solutions containing a combination of solutes that simultaneously chemically modify bacterial and viral proteins so as to disrupt their normal function and disrupt the physical integrity of bacterial and viral lipid membranes by providing destabilizing pH, disulfide bond reduction capability, lipid membrane disruption (detergent) capability, hyperosmotic stress and interference with the binding of viral or bacterial proteins to cellular surface proteins.

### CROSS-REFERENCES TO RELATED APPLICATIONS

The present application is a U.S. National Phase Application which claims the benefit of U.S. Provisional application No. Application Number 63/058,580 (filed Jul. 30, 2020 which is herein incorporated by reference in its entirety).

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### REFERENCE TO A “SEQUENCE LISTING”

Not Applicable

### FIELD OF THE INVENTION

The invention described here provides a novel method of destroying pathogenic viruses and bacteria in the human nasopharyngeal cavity and on infected wounds of the external epithelia (skin) by creating a simultaneous set of at least four physiological stressors.

### BACKGROUND OF INVENTION

The problem of prophylactic defense against serious viral and bacterial infections of the human respiratory system and antibiotic resistant bacterial infection of wounds of the epithelium of humans is an ongoing medical problem of great magnitude. Two major airborne diseases, influenza and coronavirus, have created planet wide in the last hundred years infecting hundreds of millions of people and killing tens of millions of people. In addition, airborne anthrax and smallpox have the potential to be used as biological weapons. Vaccines are available for smallpox and some strains of influenza, and antibiotics are effective against anthrax. However, it is a huge logistical challenge to create and distribute successful vaccines in a reliably timely manner to control especially deadly strains of coronavirus, influenza, or intentionally bioengineered smallpox or anthrax. This implies that wide spectrum microbial biocides that can be applied easily and quickly to both personal protective equipment, the human nasopharyngeal cavity could be a significant first line of defense. In addition safe, nontoxic wound dressings of wide spectrum microbiocidal activity and highly resistant to loss of efficacy due to microbial mutation would be of great value to medicine. Additional value would accrue if such biocides' components could be widely and cheaply produced. Such treatments should rely heavily on general biophysical and biochemical vulnerabilities of both virus and bacteria. These include the disulfide bonds of proteins, the integrity of viral envelopes and bacterial plasma membranes, the osmotic integrity of viral and cellular interiors including appropriate electrolyte composition and appropriate pH. Various extent formulations address components of this approach. The quaternary ammonium compounds (QACs) have a wide application as standalone broad spectrum anti-microbials and have low toxicity. QACs act by binding to negative sites on viral and bacterial membranes by way of the positively charged quaternary ammonium head group, then inserting a long hydrophobic tail into the hydrophobic core of the viral envelope or bacterial plasma membrane causing major disruption of the lipid bilayer including uncontrolled exchange of interior and exterior contents. Some bacteria, in particular, have developed enzymatic defenses against QACs. The modified amino acid acetylcysteine has low toxicity and is an effective disulfide bond reducing agent. It has been proposed as an antiviral. It is especially effective reducing disulfide bonds at moderately alkaline pH circa 9. High pH has been shown to reduce infectivity in some viruses and appropriate non-toxic buffers can be used for this. Hyperosmotic stress has also been shown to compromise viruses such as influenza. Multi-drug resistant bacteria, an increasingly challenging epidemic, have much greater defenses than viruses, but they, too, are vulnerable to membrane disruption, inappropriate cytoplasmic electrolytes and loss of critical protein disulfide bonds. Heretofore, topical antimicrobials have not been formulated to combine all four of these stressors: high pH, hyperosmotic stress, strong disulfide bond disruption and membrane bilayer disruption.

### BRIEF SUMMARY OF THE INVENTION

This invention is designed to be a set of broad antimicrobial topical formulations each utilizing multiple simultaneous stressors to defeat the defenses of pathogenic bacteria and viruses. Each consists of at least one component in sufficient aqueous concentration to raise the osmolality of the formulation above 300 mOsm, i.e. introducing osmotic stress. Each formulation contains at least one component to buffer the solution in the alkaline range to interfere with pathogens that require acidification of the environment to successfully infect the host. Each formulation contains at least one component that disrupts the integrity of the lipid bilayer envelope of viruses and the plasma membranes of bacteria. Each formulation contains at least one component that reduces disulfide bonds in proteins to sulfhydryl groups. The individual components of these formulations are approved for medical use as antimicrobials. These formulations combine them to achieve a unique multi-stress system designed to synergistically potentiate the stress induced by individual components

### BRIEF DESCRIPTION OF THE DRAWINGS

FIG. 1. A schematic representation of a typical multi-domain protein with a single polypeptide chain. Both the intra-domain and inter-domain native structure is stabilized by multiple disulfide bonds.

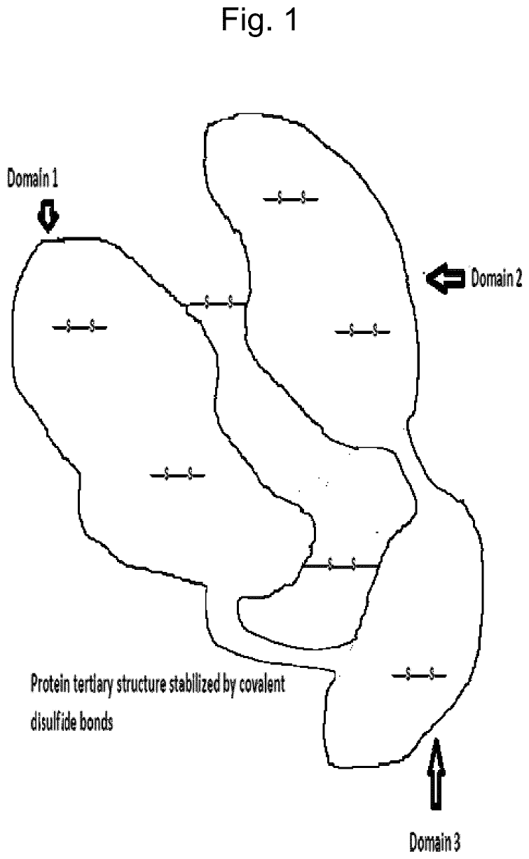

FIG. 2. A schematic representation of the protein of FIG. 1 showing that the reduction of the disulfide bonds destabilizes both the internal native structure of each domain and the native inter-domain structure.

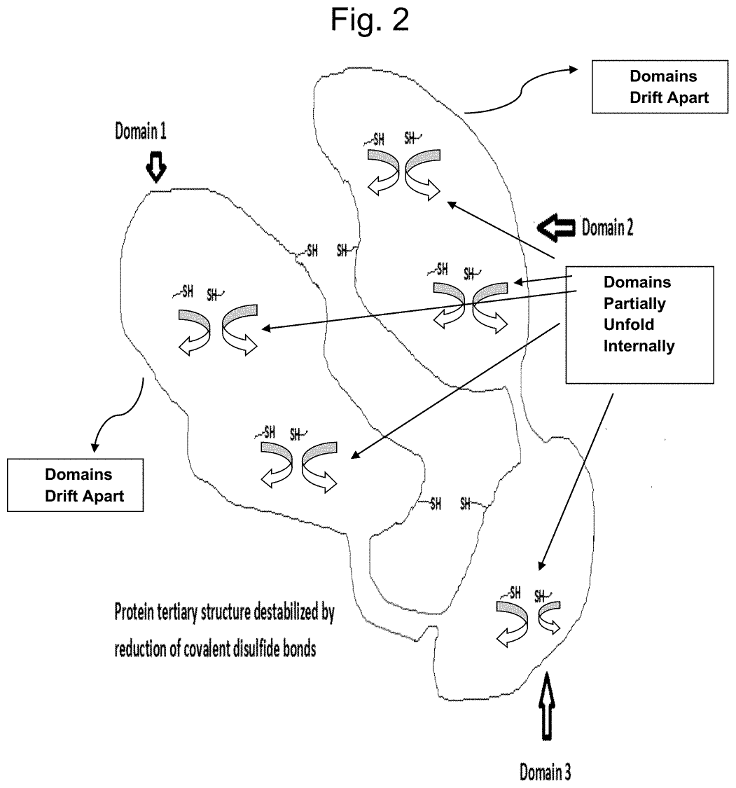

FIG. 3. A schematic representation of a typical enveloped virus illustrating the disruption of the viral lipid membrane by detergent molecules.

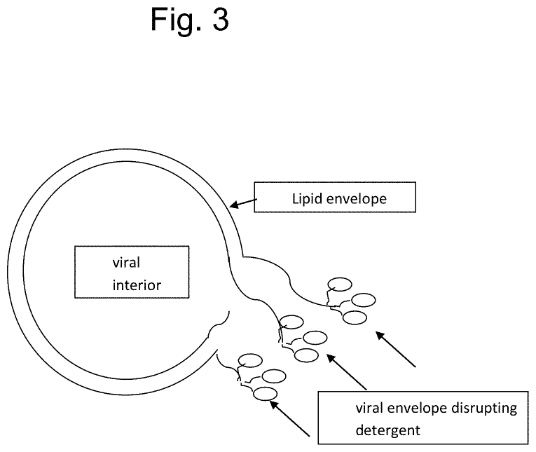

FIG. 4. A schematic representation of a virus with a spike protein that viral pathogens use to bind to target molecules on the surface of host cells as the path to enter the targeted cell. Here the spike protein is shown having its binding site blocked by a component of an anti-viral composition.

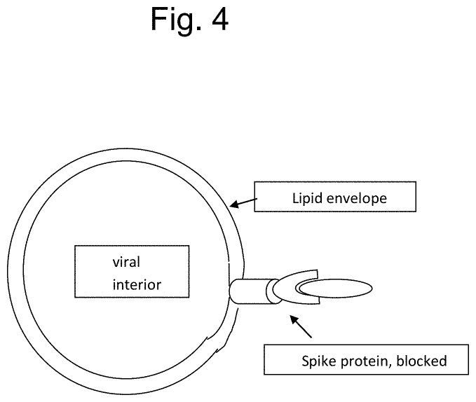

### DETAILED DESCRIPTION OF THE INVENTION

A combination of significant stressors should synergistically enhance the lethality of the individual components against pathogenic viruses and bacteria without undue mammalian cytotoxicity. The at least four stressor components of the invention are 1)hyperosmotic stress, 2)moderately extreme pH, 3)excessive disulfide bond reduction and 4)surfactant mediated membrane disruption.

Preferred solutions should exhibit both robust antimicrobial activity while minimizing the stress on nasal mucosa and injured epidermal tissue in the case of wound protection. Due to the at least one disulfide bond reducing component of the invention there should be structural destabilization of the hemagglutinin (HA) and the neuraminidase (NA) and the matrix protein M2 of influenza and the spike protein of coronavirus. The at least one surfactant stressor component should destabilize lipid bilayer membrane such as the envelope of viruses causing them to be leaky and causing exchange of vital intracapsid solutes with the external hyperosmotic alkaline solution and increasing detachment of crucial membrane bound proteins from the viral envelope as well. Studies have shown that osmotic stress alone should be an effective agent against influenza. The at least one high pH buffer component of the invention has several independent functions: it facilitates disulfide bond reduction in vulnerable pathogen proteins, especially effective in the case of acetylcysteine (pKa of SH ˜9.5) at pH>9, it creates conditions for the formation of reactive mucin thiolates in the nasopharyngeal cavity that can bind to viral SS groups and could create active thiolates on viral proteins that would attack mucin SS bonds as well hindering diffusion to nasal epithelia. Also, the at least one high pH buffer can interfere with the acidification of the endosomal space, necessary for membrane fusion following viral entry into the cellular cytoplasm by any virions that do manage to enter target cells. Wholesale disulfide bond reduction should severely structurally destabilize the aforementioned viral proteins because these molecular structures depend on numerous disulfide bonds. It should also be noted that a study has been reported that high dose intravenous acetylcysteine is directly therapeutic in pulmonary influenza, presumably due to its strong anti-oxidant properties. Among other effects of NA functional loss would be the increased capability of mucin sialic acid groups to bind all of the envelope proteins and thereby hinder diffusion to the cellular surface. Conversely, compromising the ability of NA to disable the sialic acids of dying cells from which virions are emerging should help keep them localized, hampering spread towards the lungs. In the case of SARS-coV-2 the human binding target ACE2 membrane protein itself has 3 disulfide bonds and the host coenzyme TMPRSS2 has 9, making them additional targets for destabilization. In influenza the acidification of the endosomal space, crucial to successful membrane fusion depends on the formation of a proton channel in the endosomal membrane by the viroporin M2. Reduction of the M2 disulfide bonds followed by surfactant solubilization has been shown to inactivate M2. Surfactant alone has shown strong anti-viral activity, but here it can function in several synergistic roles beyond just direct destabilization of the envelope membrane. Notably, since both spike proteins and NA are anchored in the envelope by hydrophobic sequences significant disruption of the bilayer may lead to release of these proteins from the virion. Finally, reducing mucin viscosity because of disulfide bond reduction may facilitate transport of virus particles to the gut. Another of the at least four stress components of this invention is osmotic stress. Many bacteria express high concentrations of intracellular osmolytes and are very resistant to osmotic stress alone. A classic example of a naturally occurring osmotic stressor is honey which due to its viscosity and extreme osmotic potential naturally resists the growth of microorganisms. Another example of this is the use of hypertonic NaCl to preserve meat and pickled vegetables. The sensitivity of Staphylococcus aureus (MRSA) to osmotic stress in the presence of a bactericidal surfactant has been shown to be strongly synergistic when the osmotic stress was >1 osmolar. Another stressor applied to microorganisms by this invention is the ability to disrupt the semipermeability of envelope membrane(s) in viruses and the plasma membranes of bacteria. Well known examples of membrane disrupters are the quaternary ammonium cationic detergents, such as cetylpyridinium chloride (CPC), which has been shown to be effective in concentrations as low as 30 μg/ml. Literature has also demonstrated strong synergy between hyperosmolality and membrane disruption. For example, detergent induced membrane disruption combined with the hyperosmotic electrolyte concentration of NaCl leads to significant bacterial mortality compared to detergent alone. A third stress component of this invention that has been found to impact bacterial growth and stability is pH. Studies of the sensitivity of wild type Staph aureus to quaternary ammonium compounds, such as CPC, shows a very significant increase in bacterial mortality as the external pH is raised from neutral to pH 9. If pH 9 is achieved with an effective buffer such as 2-Amino-2-(hydroxymethyl)propane-1,3-diol (Tris), this should accentuate the effect on bacterial mortality. Thus, solutions of Tris hydrochloride at pH≥9 would be antagonistic to cytoplasmic protein functions and induce leakage of vital cellular components, including solutes the bacteria use to counter hyperosmotic stress, complementing the action of other surfactant components . As mentioned above high pH also strongly facilitates the disulfide bond reduction capability of disulfide reducing agent like acetylcysteine, which studies have shown can be effective against bacterial biofilms. A fourth stressor of this invention is at least one component facilitating disulfide bond reduction in viral and bacterial proteins. A preferred compound of interest is acetylcysteine. At neutral pH, this highly non-toxic compound is a rather ineffective disulfide bond reducing agent due to its high pKa of 9.4. But at pH 9 or above, acetylcysteine is very effective because of the greatly increased proportion of the thiolate form, which serves as a very aggressive nucleophile. As a result, this compound would be expected to destabilize any bacterial proteins requiring intact disulfide bonds. In Staphylococcus aureus, which has an unusually low abundance of cytoplasmic proteins with disulfide bonds, acetylcysteine at high pH should prevent reoxidation of the disulfide bond forming DsBa analog. This, in turn, should block crucial disulfide bond formation in DsBa target proteins (secreted crucial toxins and cell binding proteins), rendering them inactive. Due to the fact that Acinetobacter and other gram negative targets have a much higher proportion of disulfide bond stabilized proteins, these bacterial pathogens should be even more susceptible to facilitated disulfide bond reduction, thus increasing the effects of acetylcysteine. Overall, large scale severing of disulfide bonds should destabilize many critical proteins in all bacterial targets.

An embodiment of the present invention consists of an aqueous solution of Tris 500 mM pH ≥9.0 and 700 μg/ml cetylpyridinium and the disulfide reducing agent acetylcysteine at 20 mg/ml

An embodiment of the present invention consists of an aqueous solution Tris 500 mM pH ≥9.0 and 1.3 mg/ml benzalkonium chloride and the disulfide reducing agent acetylcysteine at 20 mg/ml

Another embodiment of the present invention consists of an aqueous solution 1.3 mg/ml benzalkonium chloride and 100 mM tris buffer pH ≥9.0 and 20 mg/ml acetylcysteine and 400 mM NaCl, having an osmotic strength of ˜625 mOsm.

Another embodiment of the present invention consists of an aqueous solution of 0.15 mM triton x-100 and 10 ug/ml CPC and 100 mM tris buffer pH ≥9.0 and 20 mg/ml acetylcysteine and 400 mM NaCl, having an osmotic strength of ˜625 mOsm.

What is claimed is:

1) An aqueous solution consisting of at least one buffering component creating a solution pH between pH 2 and pH 10 and at least one disulfide bond reducing component capable of reducing the disulfide bonds of proteins and at least one detergent component capable of degrading the integrity of the lipid membranes of bacteria and/or viruses.

2) The composition of claim 1 wherein each of the components is present in a concentration from 10-6 molar to 1 molar.

3) The composition of claim 1 wherein each of the buffering components contains at least one functionality selected from the group consisting of amino, amido, imino, imido, carboxylic, sulfonic, phosphoric and phosphonic

4) The composition of claim 1 wherein at least one of the disulfide bond reducing agents contains at least one sulfhydryl functionality.

5) The composition of claim 1 wherein at least one of the detergent components contains at least one functionality selected from the
group consisting of amino, amido, imino, imido, quaternary ammonium, carboxylic, sulfonic, sulfate, phosphoric and phosphonic.

6) The composition of claim 1 wherein the least one of the three aqueous solutes are from a group of compounds comprising sodium bicarbonate, benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride, domiphen bromide, acetylcysteine, glutathione, dithiobutylamine and human thioreductase.

7) The composition of claim 1 applied to human nasopharyngeal cavities or epidermal wounds as an aerosol and/or lavage and and/or topical application.

8) The composition of claim 1 applied to protective masks covering the mouth and nose.

9) An aqueous solution consisting of at least one buffering component creating a solution pH between pH 2 and pH 10 and at least one disulfide bond reducing component capable of reducing the disulfide bonds of proteins and at least one detergent component capable of degrading the integrity of the lipid membranes of bacteria and/or viruses and at least one component that interferes with binding of bacterial and/or viral proteins to proteins on the surface of mammalian cells.

10) The composition of claim 9 wherein each of the components is present in a concentration from 10-9 molar to 1 molar.

11) The composition of claim 9 wherein each of the buffering components contains at least one functionality selected from the group consisting of amino, amido, imino, imido, carboxylic, sulfonic, phosphoric and phosphonic

12) The composition of claim 9 wherein at least one of the disulfide bond reducing agents contains at least one sulfhydryl functionality.

13) The composition of claim 9 wherein at least one of the detergent components contains at least one functionality selected from the group consisting of amino, amido, imino, imido, quaternary ammonium, carboxylic, sulfonic, sulfate, phosphoric and phosphonic.

14) The composition of claim 9 wherein the least one of the three aqueous solutes are from a group of compounds comprising sodium bicarbonate, benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride, domiphen bromide, acetylcysteine, glutathione, dithiobutylamine and human thioreductase.

15) The composition of claim 9 applied to human nasopharyngeal cavities or epidermal wounds as an aerosol and/or lavage and and/or topical application

16) The composition of claim 9 applied to protective masks covering the mouth and nose.

17) The composition of claim 9 wherein the at least one component capable of blocking the binding of viral proteins to cellular proteins are from a group of compounds comprising ritonavir, niclosamide, erythromycin, clarithromycin ,azithromycin, heparin, ivermectin, fidaxomycin, DX600 [amino acid sequence of Ac-GDYSHCSPLRYYPWWKCTYPDPEGGG-NH2], MLN-4760 [(S,S)-2-(1-Carboxy-2-(3-(3,5-dichlorobenzyl)-3H-imidazol-4-yl)-ethylamino)-4-methylpentanoic acid, 2(S)-(1(S)-Carboxy-2-(3-(3,5-dichlorobenzyl)-3H-imidazol-4-yl)-ethylamino)-4-methylpentanoic acid], N-(2-aminoethyl)-1 aziridine-ethanamine,
TAPI-2 [N-(R)-(2-(Hydroxyaminocarbonyl)methyl)-4-methylpentanoyl-L-t-butyl-glycine-L-alanine 2-aminoethyl amide], Emodin [6-methyl-1,3,8-trihydroxyanthraquinone], Resveratrol [trans-3,5,4′-trihydroxystilbene]

18) An aqueous solution consisting of at least one buffering component creating a solution pH between pH 2 and pH 10 and at least one disulfide bond reducing component capable of reducing the disulfide bonds of proteins and at least one detergent
component capable of degrading the integrity of the lipid membranes of bacteria and/or viruses and at least one component that interferes with binding of bacterial and/or viral proteins to proteins on the surface of mammalian cells and at least one set of enzymatic components capable of reducing the disulfide bonds of proteins.

19) The composition of claim 18 wherein the at least one component of the set of enzymatic components capable of reducing the disulfide bonds of proteins consists an aqueous solution containing thioredoxin, thioredoxin reductase and reduced nicotinamide adenine dinucleotide phosphate

20) The composition of claim 18 wherein each of the components is present in a concentration from 10-10 molar to 1 molar.

21) The composition of claim 18 wherein each of the buffering components contains at least one functionality selected from the group consisting of amino, amido, imino, imido, carboxylic, sulfonic, phosphoric and phosphonic

22) The composition of claim 18 wherein at least one of the disulfide bond reducing agents contains at least one sulfhydryl functionality.

23) The composition of claim 18 wherein at least one of the detergent components contains at least one functionality selected from the group consisting of amino, amido, imino, imido, quaternary ammonium, carboxylic, sulfonic, sulfate, phosphoric and phosphonic.

24) The composition of claim 18 wherein the least one of the three aqueous solutes are from a group of compounds comprising sodium bicarbonate, benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride, domiphen bromide, acetylcysteine, glutathione, dithiobutylamine and thioreductase.

25) The composition of claim 18 wherein the at least one component capable of blocking the binding of viral proteins to
cellular proteins are from a group of compounds comprising ritonavir, niclosamide,erythromycin, clarithromycin ,azithromycin, heparin, ivermectin, fidaxomycin, and umifenovir
